(19) United States
(12) Patent Application Publication
(10) Pub. No.: US 2019/0336374 A1
(43) Pub. Date: Nov. 7, 2019

(54) GROSSING STATION

(71) Applicant: MP Acquisition, LLC, Oak Park, MI (US)

(72) Inventor: Michael S. McClain, Waterford, MI (US)

(73) Assignee: MP Acquisition, LLC, Oak Park, MI (US)

(21) Appl. No.: 16/403,854

(22) Filed: May 6, 2019

Related U.S. Application Data
(60) Provisional application No. 62/667,788, filed on May 7, 2018.

(51) Int. Cl. A61G 13/00; G01N 33/00; A61G 13/10; A61B 16/00

(52) U.S. Cl. CPC A61G 13/0027; A61B 16/00; A61G 13/10; G01N 33/0047

(57) ABSTRACT

A grossing station for use in autopsy, necropsy, dissection, and laboratory work comprises a table and a hood connected to the table. The grossing station also comprises an integrated gas sensor system having at least one gas sensor, wherein the integrated gas sensor system is secured to a bottom surface of the hood. The integrated gas sensor system provides real time sampling and reporting of a predetermined noxious gas within a work area of the grossing station. The grossing station may also include a second gas sensor used in conjunction with the first gas sensor to allow for more accurate detection and reporting of over exposure events to a user of the grossing station.

This application claims the benefit of U.S. Provisional Patent Application 62/667,788—Filed: May 7, 2018

### BACKGROUND OF THE INVENTION

### 1. Field of the Invention

The present invention generally relates to an autopsy table and more particularly relates to a grossing station having an integrated gas sensor arranged thereon.

### 2. Description of Related Art

Autopsy tables, grossing tables, necropsy tables and trimming tables are all well known in the art. These tables are designed to accommodate a body or a part of a body while a post mortem examination is carried out or pathology is conducted on an organ or other body part. Many of these autopsy tables and/or grossing stations in the prior art were made of simple ceramic slabs with no exhaust systems. Recently these prior art autopsy tables have been made of a stainless steel having a discontinuous or perforated surface. These tables may have ventilation systems to reduce the odor associated with chemicals, natural fluids and components that are associated with an autopsy or pathology procedure. The use of ceramic slabs or stainless steel create an easy to clean surface that generally are non porous and easy to disinfect after each autopsy or pathology procedure is conducted.

Many of these prior art autopsy or grossing stations include an assortment of components used in conjunction therewith. For instance, an autopsy station may include a plurality of faucets that are used for transferring liquids, gas, or vapors to the autopsy station. Such liquids can be formaldehyde, other chemicals, water, or any other type of chemical or substance needed during an autopsy or pathology procedure. The autopsy tables of the prior art generally also include an air system that includes an air intake and air exhaust vents that may also include filters and the like to constantly clean the air and reduce odors from the autopsy table and surrounding environment. Furthermore, sinks are generally located within an autopsy table. Grating surfaces may also be located on the autopsy table for allowing fluids to be removed and collected during autopsy of the human or animal body. Furthermore, autopsy tables may also be arranged such that a gurney or cart may be used in conjunction with the autopsy table or grossing station for delivering the body or for holding the body during the autopsy procedure. The gurney or cart may be capable of being secured in position against a predetermined surface of an autopsy table and may also be inclined to any required angle necessary for the user performing the autopsy.

Prior art autopsy tables have generally been used in fixed positions in a post mortem room, hospital, laboratory or the like. Furthermore, these tables generally are fixed in a position that is convenient for a predetermined average size human to conduct the work of the autopsy or pathology procedures in a comfortable, non-stress inducing manner in a standing or seated position.

One possible problem associated with prior art autopsy, necropsy, grossing stations or tables is over exposure to formaldehyde, which generally is used during an autopsy procedure and may be harmful to the person performing the autopsy procedure. Generally, the use of formaldehyde in a pathology lab is essential and therefore, the out gas from the chemical on tissue samples and in the storage containers must be considered when grossing stations are in use. In fact, OSHA recommends that limits for formaldehyde exposure in the work place be 0.75 parts of formaldehyde per million parts of air (0.75 ppm) measured as an eight hour time weighted average (TWA) or a short term exposure limit (STEL) of two parts per million which is the maximum exposure allowed in a fifteen minute period to a person working with formaldehyde. Typically today in the prior art, a lab may utilize an exposure limit tracking badge worn directly on a person to track any known exposure to the gases being used in the laboratory. Typically, these exposure limit tracking badges are only used intermittently or infrequently and are often forgotten to be placed on the individual during the grossing and autopsy procedures. Furthermore, the badge may only report a predetermined exposure limit or it must be further analyzed to establish a trend in exposure over a longer time frame. Hence, there is no real time monitoring or otherwise specific detection of individual gas sources emitting towards the user using a grossing station during an autopsy or other pathology procedure.

Therefore, there is a need in the art for an improved grossing station that is capable of having a real time integrated gas sensor arranged thereon. There also is a need in the art for a grossing station that is capable of having gas sensor system, such that the amount of formaldehyde a worker using the grossing station is being exposed to is measured over a predetermined average window and/or over a short term exposure limit window. There also is a need in the art for a grossing station that has an integrated gas sensor that provides real time monitoring and other specific detection of individual sources of gases that may be potentially exposed to the user of the grossing station.

### SUMMARY OF THE INVENTION

One object of the present invention may be to provide a novel and unique grossing station.

Another object of the present invention may be to provide a grossing station that has an integrated gas sensor arranged on a surface thereof.

Still another object of the present invention may be to provide a grossing station that has an integrated gas sensor that is capable of real time monitoring and specific detection of individual gases which may be in or near a user of the grossing station.

Still another object of the present invention may be to provide a grossing station that monitors an average exposure to gases used in an autopsy over a predetermined time interval or a short term exposure limit to gases used in autopsy

Still another object of the present invention may be to provide a grossing station that allows for a person to track exposure to any gases in the laboratory in a real time environment and for a variety of gasses that may be used in the environment, such as any aqueous form of formaldehyde.

Still another object of the present invention may be to provide a grossing station that may alert the user of the immediate condition of exposure of any out gases from aqueous formaldehyde or any other gas and also may record the level of exposure over a predetermined time period.

According to the present invention, the foregoing and other objects and advantages are obtained by a novel design for a grossing station for use in an autopsy, necropsy or pathology procedure. The grossing station comprises a table and an overhead hood connected to the table. The grossing station also comprises a blower and ventilation system arranged on the table. The grossing station may also comprise a plurality of interchangeable plates arranged in a trough to allow for an either left handed or right handed grossing station depending on the user of the grossing station. The grossing station may also comprise a lift system to allow for the height of the grossing station to be adjusted. The grossing station may also comprise a filter hour meter, an eco mode, a remote warning system and a low air flow rate CFM warning system. The grossing station may also comprise a user configurable alarm setting to allow for individualized and predetermined alarm configurations. The grossing station may also comprise a touch screen controller that increases the reliability of the grossing station and also improves the system robustness by including manual redundancy to allow the user to manually operate different features of the grossing station. The grossing station may also comprise an improved lighting system that improves the lighting strategy by focusing on light temperature, work area light coverage and shadow reduction for the user. The grossing station may also comprise a formalin refill system that includes easy and automatic refill of the formalin system without removal of the formalin tank. The grossing station may also comprise an integrated gas sensor arranged on a predetermined surface of the grossing station. The integrated gas sensor may provide for a real time reporting of gassing from aqueous formaldehyde which may alert the use of any immediate condition of exposure to such gas. The integrated gas sensor system also may be capable of providing a record of the level of exposure to aqueous formaldehyde or other gases over any length of time, such as days, weeks, months or years.

One advantage of the present invention may be that it provides a novel and unique grossing station.

Another advantage of the present invention may be that it provides for a grossing station having an integrated gas sensor arranged thereon.

Still another advantage of the present invention may be to provide a grossing station that allows for real time reporting about gassing from aqueous formaldehyde in order to alert the user of the grossing station to the immediate condition of exposure to such gas.

Still another advantage of the present invention may be that it provides a grossing station that uses an integrated gas sensor that is also capable of recording the level of exposure over time to aqueous formaldehyde or any other gasses, such as bleach, alcohol or xylene found in the grossing station environment.

Another advantage of the present invention may be that it provides a grossing station that is capable of using a software algorithm to display sensor status or to provide a tone through a control panel arranged on the grossing station to alert the user to any exposure to aqueous formaldehyde or any other gas being monitored.

Still another advantage of the present invention may be that the integrated gas sensor may be capable of measuring aqueous formaldehyde in parts per million and through an associated methodology and software to alert the user when both long term and short term exposure limits have been exceeded.

Still another advantage of the present invention may be that methodology or software associated with the integrated gas sensor may be configured to report immediate conditions and also keep a running average for long term exposure reporting for the workers of the lab.

Another advantage of the present invention may be that it provides a grossing station that has an integrated gas sensor which uses an electrical or chemical type sensor or a semi-conductor gas sensor for detecting aqueous formaldehyde or other gases in the grossing station environment.

Other objects, features and advantages of the present invention will become apparent from the subsequent description, and appended claims taken in conjunction with the accompany drawings.

### BRIEF DESCRIPTION OF THE DRAWINGS

FIG. 1 shows a perspective view of a grossing station according to the present invention.

FIG. 2 shows a perspective view of a gas sensor for use with the grossing station according to the present invention.

FIG. 3 shows an alternate embodiment of a gas sensor for use with a grossing station according to the present invention.

FIG. 4 shows an alternate embodiment of a gas sensor for use with a grossing station according to the present invention.

FIG. 5 shows an alternate embodiment of a gas sensor for use with a grossing station according to the present invention.

FIG. 6 shows an alternate embodiment of a dual gas sensor for use with a grossing station according to the present invention.

FIG. 7 shows an alternate embodiment of a dual gas sensor used in conjunction with a manifold and valve according to the present invention.

FIG. 8 shows a partially exploded view of an alternate embodiment of a dual gas sensor for use with a grossing station according to the present invention.

FIG. 9 shows an alternate embodiment of the integrated gas sensor system for use with a grossing station according to the present invention.

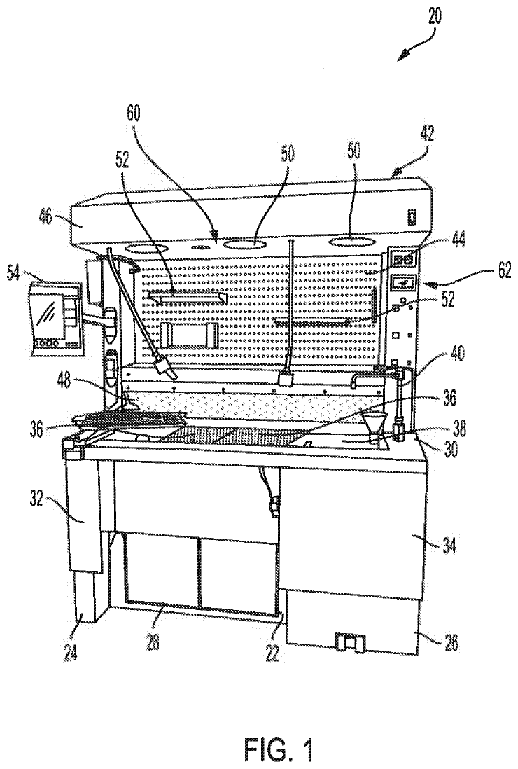

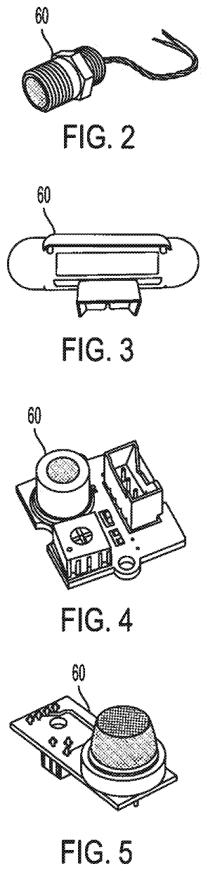

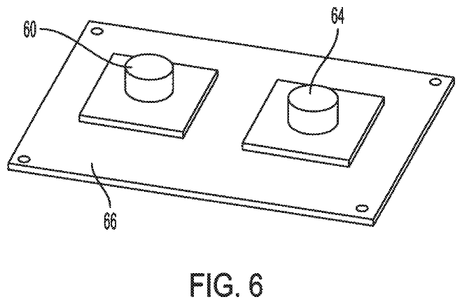

### DESCRIPTION OF THE EMBODIMENT(S)

Referring to the drawings, a grossing station 20 that is capable of being adjusted to various heights is shown according to the present invention. It should be noted that the grossing station 20 may be used on any known autopsy, necropsy, trimming, pathology, or any other procedure performed on tissues or the like all of which may be for use in hospitals, morgues, funeral homes, university laboratories, other laboratories and any other structure that can be used for examining human and animal bodies or human and animal body parts.

The grossing station 20 of the present invention includes a base 22, which generally is arranged on a floor or other surface in a lab or room in which the grossing station 20 may be used. It should be noted that it is also contemplated to attach the grossing station base 22 to a wall and elevate it off of the floor or to place it on a frame above the floor. The base 22 generally includes a first 24 and second leg 26 and a cross member arranged therebetween. The base 22 may have any known predetermined height and width, Each of the legs 24,26 generally may have a square or rectangular shape when viewed from the side. The base 22 may have an appropriate pad arranged at a bottom surface of the legs which engages with the floor or a surface of the lab in which the grossing station 20 is arranged. The base 22 may include an opening in the cross member thereof which may have a door 28 arranged in the opening. The door 28 in one contemplated embodiment is a sliding door, the door 28 arranged as shown in the figures, is a swinging door and/or a sliding door. However, the door may be arranged on both portions thereof to cover the opening in the base 22. It should be noted that any other type of door, such as a swing door, panel door, folding door, or the like may be used in the base 22. It should be noted that a shelf may also be arranged within the opening of the base 22 behind the door 28. The shelf may be made of any known material and may have any known dimensions. The shelf, which may be a slidable shelf, generally may have a rectangular bottom surface that may or may not have a perforated panel arranged thereon. A lip may be formed at the front edge of the sliding shelf or along the entire outer periphery of the sliding shelf. It should be noted that the sliding shelf may have any other known shape other than rectangular depending on the design requirements of the grossing station 20. In one contemplated embodiment the sliding shelf may be used to hold and store formalin or other chemicals used during autopsy, necropsy or pathology procedures. This may allow for the formaldehyde or formalin to be passed through associated tubing from the storage container up to the surface of the grossing station table 30 and then return to a holding or waste container which is located outside of the sliding shelf or on the sliding shelf itself. A plurality of connections may end in faucets, spickets, spray guns, or the like on the grossing station work surface to allow for the autopsy procedure being conducted to access the necessary chemicals for preparing, storing and examining the tissues thereon. It should be noted that the base 22 is generally made of a metal material, however any other ceramic, composite, natural material, plastic or the like may be used for the base 22 and the shelf arranged therein. Furthermore, the panels used on the door 28 of the base 22 generally have a glass material arranged therein to allow for viewing of the contents of the shelf. However, any other known plastic, ceramic, composite, natural material, metal or the like may also be used for the door 28 or doors arranged on the base 22 in front of the shelf.

The grossing station 20 may also include a table or work surface area 30 arranged over a top portion of the base 22. The table 30 may have a generally rectangular and flat work surface arranged on the top end thereof and a first 32 and second leg 34 arranged on the sides thereof. The first 32 and second legs 34 are arranged over the first and second legs 24,26 of the base 22. A lifting mechanism may be arranged between the base 22 and the table 30 of the grossing station 20. This may allow for the table 30 to be adjusted to any number of predetermined heights to accommodate different size users conducting the pathology procedures on the grossing station 20. It should be noted that any type of lifting mechanism may be used on the grossing station 20 of the present invention, such as but not limited to a linear actuator, scissor actuator, screw actuator or any other lifting device, electronic, fluid driven, gas driven, air driven, electrical or mechanical driven, may be used to move the table 30 with relation to the base 22 to adjust the grossing station work surface to a predetermined height. The table 30 also may include a trough arranged in a top surface thereof. The grossing station 20 also may include a switch arranged on a front surface of one of the legs or other surface of the grossing station table 30 that may allow for the upward and downward movement of the work surface of the table 30 with relation to the base 22 and floor or surface on which the grossing station 20 is arranged. It should be noted that the associated electronics are in electrical communication with the switch and the lifting mechanism and to the power grid into which the grossing station 20 is connected. Other switches also may be arranged on the table 30 at predetermined positions or any other surface of the grossing station 20 to operate the blower and ventilation system, lights, computers, etc., for the grossing station 20.

The table 30 of the grossing station 20 may include a plurality of removable and interchangeable grid plates 36 which may be arranged and fit securely into the trough of the table 30. The grid plates 36 generally are rectangular or square however any other shape grid plate 36 may be used depending on the design of the grossing station table 30. The grid plates 36 may be of any known design, but generally include at least a solid plate design, a perforated plate design, and a sink 38. This may allow the user of the grossing station 20 to customize the work surface for the grossing station 20 per their individual requirements. It is contemplated to use the grid plates 36 in any combination of perforated grid plates, solid plates and sinks, a combination of sinks and perforated plates, a combination of sinks and solid plates, a combination of all sinks, a combination of all perforated plates and one sink, etc. Any known configuration may be used with the interchangeable grid plates 36. Generally, the interchangeable grid plates 36 may have a rounded front edge to help create an ergonomic edge for use by the user of the grossing station 20. The grid plates 36 may interact with a first and second shoulder arranged near a top surface of the grossing station table 30 at the trough edges. The sink 38, which may be used as one of the grid plates 36, may have a drain hole that interacts with the trough and/or a drain that is connected to the trough which then drains to the sewage system or other storage area of the building into which the grossing station 20 is arranged. These connections are made by any known piping used in the plumbing industry. It should be noted that the grossing station 20 may be set up in either a right handed sink setup or a left handed sink setup depending on the dominant hand of the user of the grossing station 20. Generally, connected to the top surface on either one or both sides of the table 30 may be a water faucet 40 which may allow for water to be used on either side of the grossing station 20 and to be drained into either a left handed or right handed sink 38.

The table 30 also may include a blower and ventilation system which may move air across the entire table including any sinks 38 arranged within the top surface of the table 30 in any known direction, such as a forward to rear direction, bottom to top direction, etc. The ventilation system may include any number of ventilation ducts and blowers arranged in any predetermined manner.

The grossing station 20 also may include a hood 42 that is secured to a top surface of the table 30 of the grossing station 20. The hood 42 generally may have two side walls and/or partial sidewalls, a rear wall 44 and a top member 46. The top member 46 when viewed from above generally has a rectangular shape. The hood 42 may also include a flexible bellow duct, an access panel, a filler retention bracket, and back draft grill 48. It should be noted that any necessary lighting 50 and associated electronics to control the lighting may be arranged on surfaces of the hood 42 or on the walls of the hood 42. The rear wall 44 of the hood 42 also may have a plurality of shelves 52 arranged thereon for holding supplies, tools, computer equipment 54, etc., or the like necessary for the pathology procedure being performed at the grossing station 20. According to the figures, the hood 42 may have a perforated back wall 44 that allows for the placement of shelves 52, hooks, or other components in a variety of configurations depending on the user's requirements. The hood 42 is generally made of a stainless steel material as are all or most of the other parts, but may be made of any other metal, plastic, ceramic, composite, or natural material depending on the design requirements and the environment in which the grossing station 20 may be used. In one contemplated embodiment the use of sliding side glass panels may allow for the grossing station 20 to create a fume hood, which may be certified to remove all fumes during the autopsy, necropsy or pathology procedure thus meeting more stringent standards required in some laboratories and hospital environments.

In operation, it should be noted that all of the necessary piping, plumbing, and electrical connections are secured either to a surface of the hood 42, the table 30 or base 22 of the grossing station 20 and are connected to the building systems in which the grossing station 20 is arranged. Copper tubing, rubber tubing, any necessary electronic circuitry, switches, and any known pumps may be used to move the table 30 with relation to the base 22 and to move the air via the blower and ventilation system into either the outside atmosphere, a storage area or into a filter mechanism. Any known fasteners and holders, such as clips, rods, bands, screws, nuts, bolts, etc., may be used to secure the piping and electrical components to the surfaces of either the table 30, base 22 or hood 42 of the grossing station 20 according to the present invention.

As shown in the attached Figures, the grossing station 20 of the present invention also may improve the users experience by including a filter hour meter thereon. The filter hour meter may have an associated algorithm or software associated therewith to allow for changing of the air filter when deemed necessary. The grossing station 20 may also include an eco mode system that may allow for saving of energy by detecting the presence of a user at the station and turning off all unnecessary lights 52 and other electronic components when the user leaves for a predetermined amount of time. The grossing station 20 of the present invention also may have a remote warning system that may monitor the formalin tank and display a warning indicator on the grossing station 20 when the tank is empty. The remote warning system may also alert the user when the formalin waste tank is full and must be replaced. The grossing station 20 may also include an airflow monitoring system, such as but not limited to a low cubic feet per minute (CFM) or linear feet per minute (LFM) warning system that may monitor the air flow rate and allow the user of the grossing station 20 to input a lower threshold limit that may be used to set off an alarm if the CFM or LFM drops below a set predetermined low limit. The CFM or LFM warning system may be arranged and controlled via any known software. The grossing station 20 also may include within its electronic control and monitoring system controller, a software control system that may allow for the user to configure the warnings and alarms to their predetermined or preferred requirements for each user, The grossing station 20 may also include an electrical system that uses a touch screen controller, which uses a higher level of complexity. The grossing station 20 of the present invention may also include an improved lighting system that may include improved strategies that focus on the light temperature (color), the work area light coverage and shadow reduction as they impact the grossing station 20 user's experience. The grossing station 20 of the present invention may also use a unique formalin refill system. The formalin refill system may allow the user to refill the tank without having to remove the tank from the grossing station 20.

The grossing station 20 of the present invention may also include an integrated gas sensor system arranged on the grossing station 20. The use of the integrated gas sensor system may monitor the use of formaldehyde (CH₂O) in the lab. Formaldehyde is an essential chemical that is used in autopsy, necropsy and pathology procedures. Formaldehyde produces an outgas from the chemical on tissue samples and from the storage containers and must be considered and monitored when using a grossing station 20. The federal OSHA guidelines recommend that limits for formaldehyde in the workplace be approximately 0.75 parts formaldehyde per million parts of air (0.75 ppm) measured as an eight hour time weighted average (TWA) or a short term exposure limit (STEL) of two parts per million which is the maximum exposure allowed in a fifteen minute period. Therefore, there must be an integrated gas sensor 60 that is capable of detecting at least two parts per million of formaldehyde or other gas in air in real time. The integrated gas sensor system of the present invention may use a gas sensor 60 that is mounted or secured on the grossing station hood 42 or table 30 in any known position. In one contemplated embodiment, the gas sensor 60 is mounted at or in an outer surface of the hood 42 directly adjacent or near where the head of a user of the grossing station 20 may be arranged. It should be noted that the gas sensor 60 may be mounted on any other portion or surface of the table 30 or hood 42 of the grossing station 20. Generally, the gas sensor 60 may be any type of electro chemical cell sensor or a semiconductor gas sensor, such as those typically used for measuring volatile organic compounds (VOC) in the industry. It should be noted that any other known gas sensor may also be used. In one contemplated embodiment, the gas sensor 60 must be capable of measuring VOC's in the 0-2 part per million detection range. However, the gas sensor 60 may be able to measure in any known range such as but not limited to zero to 100,000 part per million, etc. It should be noted that the gas sensor 60 used in the integrated gas sensor system of the present invention is capable of providing a real time reporting of out gassing from aqueous formaldehyde or any other known gas. Aqueous formaldehyde may have at least the following forms, but not limited to these forms: formalin, methanol, methyl aldehyde, methylene glycol, methylene oxide, morbicid, paraform and superlysoform, etc. The gas sensor 60 is capable of detecting these aqueous formaldehyde forms and may be able to alert the user of the grossing station 20 to the immediate condition of exposure to such gasses. It should be noted that any gas sensor 60 identified above may be used in the present invention and a variety of typical gas sensors 60 that may be used with the integrated gas sensor system of the present invention are shown in the figures. The gas sensors 60 are capable of monitoring and sampling gasses near the user's head during grossing operations. The gas sensor 60 may be secured to or arranged in either the front edge of the hood 42 or on an extended support arm. The gas sensor 60 may also have sensor detection capabilities that allow it to alert the grossing station user of other common gas concentrations typically used in grossing operations, such as bleach, alcohol, xylene, etc.

The gas sensor 60 generally may be arranged on or secured to the grossing station 20, via any known bracket or fastening technique, at the underside front edge of the hood 42, such that the gas sensor 60 is located at or near the position of the user's head while working at the grossing station 20. It is also contemplated that a support arm may either extend in a downward direction from a bottom surface of the hood 42 of the grossing station 20 or from the back wall 44 of the hood 42 of the grossing station 20 and be secured via any known fastener, brackets or other fastening methodology such that the arm would be placed at or near a position where the user's head may be adjacent to the end of the support arm, thus allowing for the gas sensor 60 to be placed in an area generally associated with the head of the user while working at the grossing station 20. It should be noted that any other methodology for connecting a gas sensor 60 other than brackets, such as any mechanical fastening or chemical fastening technique may be used to secure the gas sensor 60 to the underside of the hood 42 of the grossing station 20, any other surface of the grossing station 20, to a support or to an end of the support arm that is connected to any surface of the grossing station 20. It should be noted that gas sensor 60 may be arranged in a predetermined orifice of the hood 42, table 30 or base 22 or connected directly to an outer or inner surface thereof. It is also contemplated to use multiple gas sensors 60 arranged at different areas on the grossing station 20 to allow for multiple sampling of the air in order to detect aqueous formaldehyde outgases in the grossing station environment. All of the associated electronics and electrical wiring associated with the gas sensors 60 may be connected to the overall electrical system of the grossing station 20 and then derive power from the grossing station 20 itself or from the building in which the grossing station 20 is arranged. In one contemplated embodiment, a control panel or display device 62 may be arranged on or near the back wall or side wall of the hood 42 of the grossing station 20. The display 62 may be connected via any known fasteners or brackets to any surface or orifice of the hood 42 or any other portion of the grossing station 20 to allow for the user of the grossing station 20 to view the gas sensor status and/or receive a visual or audible alarm or warning that aqueous formaldehyde gas has been detected in an amount greater than normal in the grossing station 20 work area. The display 62 may be any type of screen, such as an LED screen, LCD screen, or any other known display screen capable of displaying a visual warning. The display 62 may also include a speaker or other type of warning device that may provide an audible alarm of some type to alert the user of the grossing station 20 to exposure to aqueous formaldehyde gasses. It should be noted that the speaker may be a separate component from the display 62.

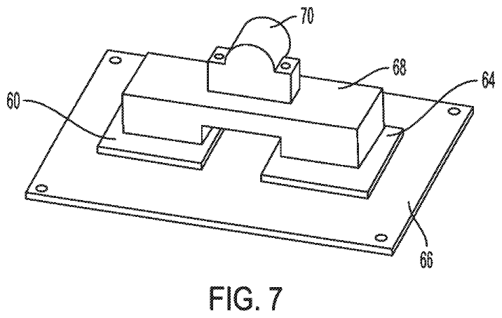

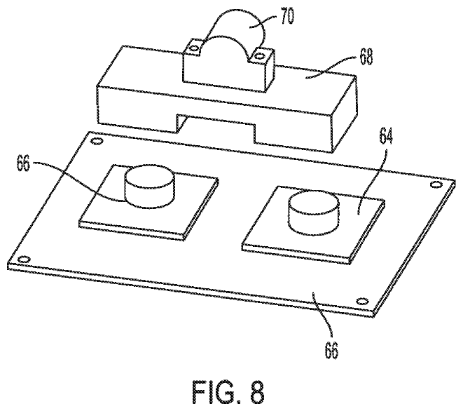

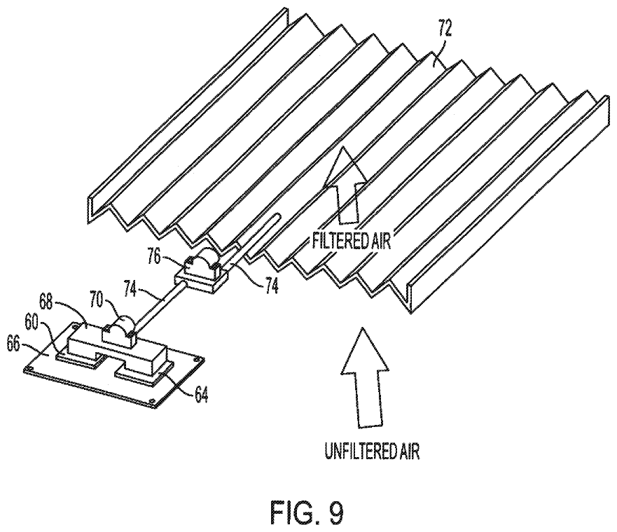

An alternate embodiment of the grossing station 20 having an integrated gas sensor system may include a first gas sensor 60 used in conjunction with a second gas sensor 64. This alternate embodiment, as shown in FIGS. 7 through 9, may generally include a circuit board 66, wherein that circuit board 66 has the first gas sensor 60 and a second gas sensor 64 arranged on one side thereof. It is also contemplated to have one of the gas sensors 60,64 arranged on opposite sides of the circuit board 60 or to have more than two gas sensors used with respect to the integrated gas sensor system. The gas sensors 60,64 may be the same as those described above, however due to the shortened life of chemical sensor cells in heavier exposure situations, a way to limit the effect of decay on the gas sensor 60 needs to be developed. The use of a first and second gas sensor 60,64 with the integrated gas sensor system may alleviate the effect of the decay. It should be noted that it is also contemplated to use the first and second gas sensor 60,64 alone and arranged on the circuit board 66 or any other surface of the grossing station 20. In this stand alone set up, the integrated gas sensor system methodology may allow for either the first gas sensor 60 or second gas sensor 64 to be active in detection of noxious gasses. The gas sensor 60,64 that is turned off may have a far lower decay rate than the gas sensor 60,64 that is active in detecting noxious gasses.

As shown in the drawings, another contemplated embodiment may have a manifold 68 arranged over the first and second gas sensor 60,64 of the integrated gas sensor system. The manifold 68 may have any known shape which allows for either the first gas sensor 60 or second gas sensor 64 to be active in detecting a predetermined noxious gas that is at or around the grossing station 20. In one contemplated embodiment, a valve 70 is arranged on the manifold 68 in order to allow for either the first gas sensor 60 or second gas sensor 64 to be the main or primary sensor used in the detection of noxious gasses around or at the grossing station hood and table. It should be noted that it is also contemplated to use just a valve 70 alone without the use of a manifold 68 arranged between the first and second gas sensor 60,64 of the integrated gas sensor system. The valve 70 may be installed on an inlet of the first gas sensor 60 which may enable the integrated gas sensor system to switch off a port of the inlet to the environmental source when gross exposure limits to noxious gas are met and then periodically open the valve 70 to sample the environment until the heavy gas conditions dissipate. Another contemplated embodiment would be to use the valve 70 in combination with a first and a second gas sensor 60,64 such that the system is tailored to handle heavy environmental conditions. For instance, the valve 70 could simply turn on the second or back up gas sensor 70 when heavy limits of gas have deactivated the first or primary gas sensor 60 in the integrated gas sensor system. It is also contemplated to use a total on or total off methodology with a first and second gas sensor 60,64 without any type of valve, wherein the primary or first gas sensor 60 after depletion thereof would be turned off and the integrated gas sensor system would turn on the second gas sensor or back up sensor 64 to continue on with the detection of noxious gasses in or around the grossing station. It should be noted that without a valve the backup gas sensor 64 in an unpowered state has very little decay of the chemical compounds thereon.

In another contemplated embodiment, after powering on the first gas sensor 60 may be used primarily to detect an event such as an over exposure to a predetermined noxious gas and stay in constant communication with the integrated gas sensor system methodology and hence to the user interface of the grossing station 20. In this case, the second gas sensor 64 would only be turned on when the first gas sensor 60 has reached its expiration via depletion of the chemical elements thereon. The circuit board 60 of the integrated gas sensor system would then be able to communicate to the user interface of the grossing station 20 that the second gas sensor 64 is operating in a back up mode and would need to be replaced after a count down of so many hours has elapsed once the back up or second gas sensor 64 is put into operating mode. In another contemplated embodiment, the circuit board 66 of the integrated gas sensor system may be outfitted with a valve 70 and manifold 68 that may allow switching back and forth between the first gas sensor 60 and the second gas sensor 64. This may be configured where the first gas sensor 60 is used primarily and the second gas sensor 64 is switched on or exposed to the environment when the first gas sensor 60 expires. Furthermore, it is also contemplated that the first gas sensor 60 may be outfitted with a higher sensitivity sensor while the second gas sensor 64 may be outfitted with a gross measurement sensor. This may allow the valve 70 and manifold 68 to be used such that when the first gas sensor 60 detects an event then the second gas sensor 64 may be used to monitor the event in a gross measurement mode thus protecting the higher sensitivity first gas sensor 60 from over decay due to the over exposure event currently occurring. Therefore, any combination of a first, second and/or any number of gas sensors 60 may be used with or without individual valves and/or a manifold in a variety of real time gas detecting and warning systems according to the present invention.

The integrated gas sensor system of the present invention may have another contemplated embodiment, wherein the sensor 60 may be used in conjunction with a filter 72 of the table 30 and hood 42. The sensor 60 may determine if the filter 72 has expired from depletion or over exposure to noxious fumes, smoke, etc. In this alternate embodiment, a siphon tube 74 is arranged between the exhausted air, either in an exhaust pipe or in the stream of exhausted air exiting from the hood 42 or table 30 and one of the gas sensor 60 on the other end. It should be noted that the siphon tube 74 may have any known length and diameter. Also, the tube may be made of any known material. Furthermore, it should be noted that the end of the siphon tube 74 taking a sample in the exhausted air from the hood 42 or table 30 may be located on the filter side of the air that is being exhausted from the hood 42 or table 30 of the grossing station 20 according to the present invention. This may allow for the sensor 60 to determined the amount of gas or other noxious fumes being detected in the filtered air and hence being captured by the filter 72 arranged between the first end of the siphon tube 74 and the second end of the siphon tube 74. The tube 74 may also have a second valve 76 arranged between its first end and second end. Once the sampled air on the filtered side of the filter 72 within the exhaust air stream of the grossing station 20 senses or reaches a predetermined value it may alert the user of the grossing station 20 that the filter 72 has been depleted and is dirty and must be changed within either a set amount of time or immediately. It should be noted that any type of filter medium may be used in conjunction with the blower and ventilation system of the present invention.

The integrated gas sensor system may have an associated methodology in the form of an algorithm or software associated therewith. The gas sensor 60 may be capable of measuring any type of aqueous formaldehyde outgas or any other type of gas in parts per million. The associated methodology may be capable of developing a warning system to alert the user of the grossing station 20 when either a short or long term exposure limit has been exceeded. In the methodology predetermined limits may be set for both short term exposure limits which in one contemplated embodiment should not exceed two parts per million and time waited average or long term exposure limits which should not exceed 0.75 parts per million. It should be noted that any other known limit values may also be used. The methodology may sample the gas sensor 60 at predetermined intervals to determined if aqueous formaldehyde or any other gas has been detected by the gas sensor 60. The methodology may be able to report immediate conditions of over exposure and also keep a running average for long term exposure and then reporting such exposure to the user of the grossing station 20 and the manager of the lab in which the grossing station 20 is arranged. This allows the user and/or lab management to monitor and correct behavior or procedures in the grossing environment to reduce exposure to aqueous formaldehyde and other gasses being monitored. The methodology may also be capable of other monitoring and reporting statistics based on the sampling times and limits of exposure to specific gases allowed by OSHA or other governing bodies.

The present description is for illustrative purposes only and it should not be construed to limit the present invention in any way. Thus, a person skilled in the art will appreciate that various modifications might be made to the present and disclosed embodiments without departing from the scope and spirit of the present invention, which is defined in terms of the claims below. Other aspects, features, and advantages may be apparent upon an examination of the attached drawing figures and appended claims.

What is claimed is:

1. A grossing station, said grossing station comprising:
a table;
a hood connected to said table; and
an integrated gas sensor system having at least one gas sensor, said integrated gas sensor system is secured to a surface of said hood, said integrated gas sensor system provides real time sampling and reporting of noxious gas within a work area of the grossing station.

2. The grossing station of claim 1 further comprising a blower and ventilation system.

3. The grossing station of claim 1 wherein said predetermined noxious gas is aqueous formaldehyde.

4. The grossing station of claim 1 further comprising a second gas sensor arranged near said gas sensor.

5. The grossing station of claim 4 further comprising a manifold, said manifold is arranged between said gas sensor and said second gas sensor.

6. The grossing station of claim 5 further comprising a valve secured to said manifold, said valve allows switching between said gas sensor and said second gas sensor.

7. The grossing station of claim 1 wherein said integrated gas sensor system measures volatile organic compounds in an approximate zero to a predetermined upper limit parts per million range.

8. The grossing station of claim 1 wherein said gas sensor is an electro chemical cell sensor or semi-conductor gas sensor.

9. The grossing station of claim 1 wherein said integrated gas sensor system having a circuit board, said gas sensor is arranged on or electrically connected to said circuit board.

10. The grossing station of claim 1 further comprising a control panel arranged on or within said hood or said table.

11. The grossing station of claim 10 further comprising a display arranged on a surface of said hood or said table, said display conveys a status of said gas sensor to a user of the grossing station.

12. The grossing station of claim 11 wherein said display has an audible or visual alarm for alerting the user of a presence of said predetermined noxious gas.

13. The grossing station of claim 4 wherein said gas sensor is a high sensitivity sensor and said second gas sensor is a gross measurement sensor.

14. The grossing station of claim 4 wherein said gas sensor is a primary gas detector and said second gas sensor is a backup detector if said gas sensor fails or is depleted.

15. The grossing station of claim 1 further comprising a siphon tube, said siphon tube is arranged between said at least one gas sensor and an exhaust airflow on a filtered side of said air flow.

16. A method of detecting noxious gases at or around a grossing station during an autopsy, necropsy, dissection or laboratory work, said method comprising the steps of:
installation a gas sensor on a table or hood of the grossing station;
sampling for a predetermined noxious gas with said gas sensor;
determining if said sampled predetermined noxious gas is greater than a predetermined limit for exposure thereto; and
reporting in real time to a user of the grossing station an over exposure to said predetermined noxious gas.

17. The method of claim 16 further comprising a second gas sensor, said second gas sensor is a back up sensor or a gross measurement sensor.

18. The method of claim 16 further comprising the steps of monitoring exposure to said predetermined noxious gas over a predetermined long term and reporting said long term exposure to a user, lab manager or predetermined governing body.

19. The method of claim 16 wherein said predetermined limit is approximately two parts per million for a short term exposure or approximately 0.75 parts per million for a long term exposure.

20. The method of claim 16 wherein said step of reporting includes using an alarm to alert the user of said over exposure, said alarm is visual and/or audible.

21. The method of claim 17 further comprises the step of switching between said gas sensor and said second gas sensor for detecting said predetermined noxious gas.
